(19) United States
(12) Patent Application Publication
(10) Pub. No.: US 2021/0308249 A1
(43) Pub. Date: Oct. 7, 2021

(54) VLP FORMULATIONS

(71) Applicant: TAKEDA VACCINES, INC., Cambridge, MA (US)

(72) Inventors: Shiro ISHII, Cambridge, MA (US); Yumiko ISHII, Cambridge, MA (US)

(21) Appl. No.: 17/269,148

(22) PCT Filed: Aug. 19, 2019
(86) PCT No.: PCT/US2019/047055

§ 371 Date: Feb. 17, 2021

(30) Foreign Application Priority Data
Aug. 20, 2018 (EP) 18189754.7
Aug. 23, 2018 (EP) 18190580.3

(51) Int. Cl. A61K 39/12; A61K 9/00; A61K 47/69; A61K 47/61; A61P 31/14

(52) U.S. Cl. CPC A61K 39/12; A61K 9/006; A61P 31/14; A61K 47/61; A61K 47/6901

(57) ABSTRACT

The present invention relates to an aqueous composition comprising water, virus like particles, at least one amino group containing mucoadhesive and at least one buffer. In certain embodiments, the virus like particles have a size average of no more than about 4 times the size average of a comparison sample of virus like particles without mucoadhesive in water. In certain embodiments, the virus like particles have a size average of no more than about 400 nm. In certain embodiments, the virus like particles have a positive zeta potential.

### TECHNICAL FIELD OF THE INVENTION

The present invention relates to vaccines, particularly to oral vaccines. In particular, the present invention relates to vaccines effective against Norovirus and to such vaccine compositions.

### BACKGROUND OF THE INVENTION

Administration via injection is the most commonly used administration method for vaccine treatments, for example via intramuscular injection or via subcutaneous injection. However, administration by injection has a number of disadvantages, for example: the risk of a needle stick injury, the risk of infection caused by re-using needles and that trained personnel are required to carry out the vaccination. Administration via injection also causes pain; therefore, alternative administration methods are preferable for patients, especially for infants, and may lead to greater compliance.

Norovirus is one of the most common causes of viral gastroenteritis worldwide. Noroviruses are highly contagious and can be transmitted through contaminated food, contaminated water or by person-to-person contact. Furthermore, outbreaks of Norovirus can lead to complications in the elderly, in children and in immunocompromised individuals.

Virus-like particles (VLPs) are non-infectious particles, which resemble viruses. The particles (e.g. Norovirus virus-like particles, NV-VLPs) are morphologically identical to the native capsids. VLPs are non-infectious because they contain no viral genetic material. Consequently, VLPs mimic the functional interactions of the virus with cellular receptors, thereby eliciting an appropriate host immune response while lacking the ability to reproduce or to cause infection. VLPs used as vaccine antigens often elicit strong immune responses due to the ability of the mammalian immune system to recognize particles on a nanometer scale, which corresponds with the size range of viruses.

Vaccine formulations including virus-like particles (VLPs) have been developed as formulations suitable for administration via injection. In U.S. Pat. No. 9,308,249 and US 2010/0266636 in particular dry particulate intranasal VLP formulations have already been developed for vaccination against the Norovirus. However, there is still a need for formulations and methods for oral vaccination.

Oral vaccination forms are desirable, since they can be administered easily to patients without pain and do not have any of the other disadvantages associated with administration via injection. Oral vaccination may even be more convenient than administration via the intranasal route. However, delivery of vaccine drug substances via an oral route can be challenging; particularly due to gastrointestinal barriers, as drug substances required for vaccination are often unstable in the gastrointestinal tract. These molecules may, for example, be unstable towards the action of proteases and/or unstable in the highly acidic environment of the stomach.

In addition, vaccines often contain toxic adjuvants such as alum, which are used to enhance the immune response to vaccine formulations. Because of the toxicity associated with these adjuvants there is interest in removing them from vaccine formulations.

### OBJECTS AND SUMMARY OF THE INVENTION

It is an object of the present invention to provide aqueous compositions containing virus like particles (VLPs), in particular aqueous compositions for eliciting protective immunity to a viral infection in a mammal.

It is a further object of the present invention to provide an oral vaccine formulation, wherein the formulation resists the gastrointestinal barriers of the mammalian digestive tract and is able to deliver VLPs to the target immune cells in the gut, thereby inducing a desirable immune response. In particular, wherein the compositions when used as an oral vaccine leads to a comparable or enhanced immune response in comparison to administration via injection.

The above objects are achieved by embodiments of the present invention as described and claimed herein.

The present invention is, therefore, directed to an aqueous composition comprising:
water,
virus like particles,
at least one amino group containing mucoadhesive, and
at least one buffer optionally with a pH of less than about 6,
   wherein the virus like particles have a size average of no more than about 4 times the size average of a comparison sample of virus like particles without said mucoadhesive in water, and/or
   wherein the virus like particles have a size average of no more than about 400 nm.

In a certain aspect the aqueous composition of the paragraph above, wherein the at least one amino group containing mucoadhesive has a pKa of less than about 7, and wherein the pH of the aqueous composition is at least about 1 pH unit more acidic than the value of the pKa of the mucoadhesive.

In a certain aspect, the invention is directed to a dried form of such a composition.

The present invention is further directed to a process of preparing an aqueous composition comprising:
water,
virus like particles,
at least one amino group containing mucoadhesive, and
at least one buffer optionally with a pH of less than about 6,
   wherein the virus like particles have a size average of no more than about 4 times the size average of a comparison sample of virus like particles without said mucoadhesive in water, and/or
   wherein the virus like particles have a size average of no more than about 400 nm, the process comprising at least the following steps:
(a) providing virus like particles,
(b) providing at least one amino group containing mucoadhesive in a buffered aqueous composition with at least one buffer, optionally with a pH of less than about 6, to obtain a mucoadhesive containing aqueous composition,
(c) adding the virus like particles to the mucoadhesive containing aqueous composition.

In a certain aspect the process of the paragraph above wherein, the at least one amino group containing mucoadhesive has a pKa of less than about 7, and wherein the pH of the aqueous composition is at least about 1 pH unit more acidic than the value of the pKa of the mucoadhesive. In said certain aspect the process of the paragraph above wherein, step (b) involves providing at least one amino group containing mucoadhesive in a buffered aqueous composition with a pH of at least about 1 pH unit more acidic than the value of pKa of the mucoadhesive, to obtain a mucoadhesive containing aqueous composition.

The present invention is also directed to a product obtainable by the said process described above.

The present invention is further directed to such compositions for use in a treatment for eliciting protective immunity to a viral infection in a mammal, in particular for use in oral vaccination, comprising methods of treatment and uses for the manufacture of a corresponding medicament.

### BRIEF DESCRIPTION OF THE DRAWINGS

FIG. 1: A schematic diagram of the chitosan coated virus like particles (VLPs) of this invention.

FIG. 2: The average size of chitosan coated VLPs in buffers of different pH corresponding to Example 1.

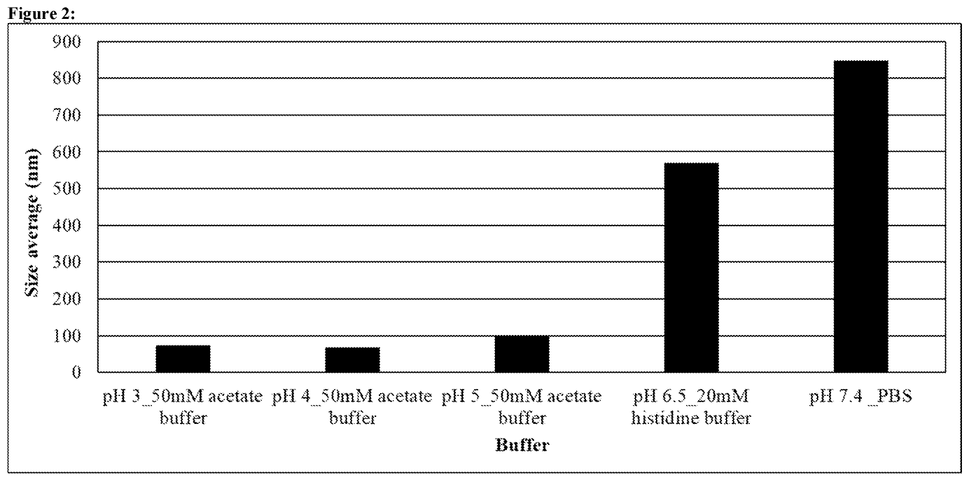

FIG. 3: The zeta potential of chitosan coated VLPs in buffers of different pH corresponding to Example 1.

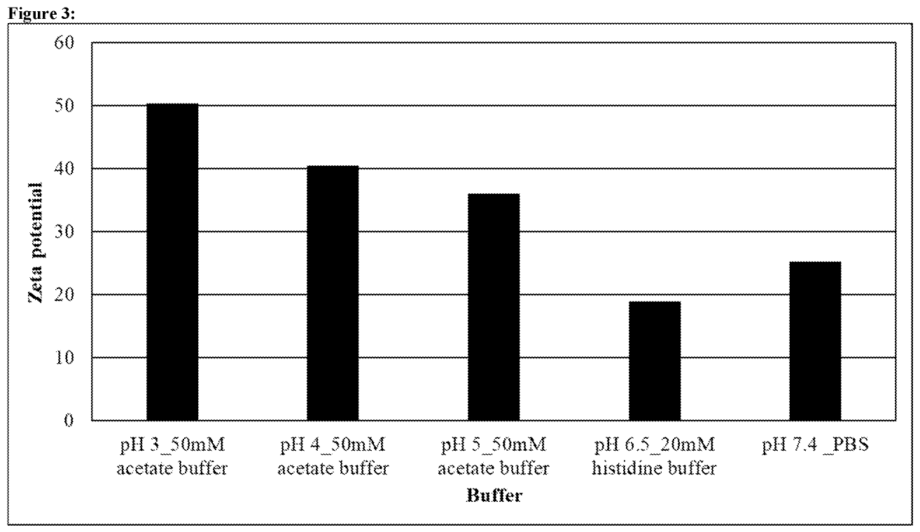

FIG. 4: The serum IgA titers in a murine model at different pHs corresponding to Example 5.

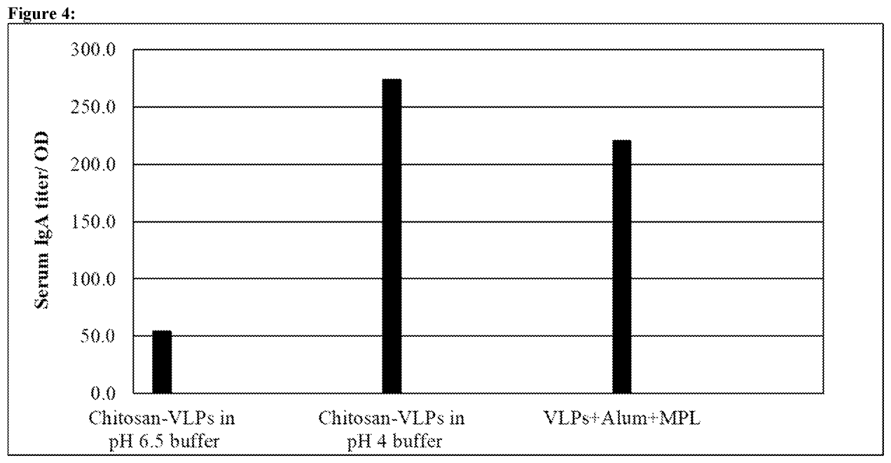

FIG. 5: The fecal IgA titers in a murine model at different pHs corresponding to Example 5.

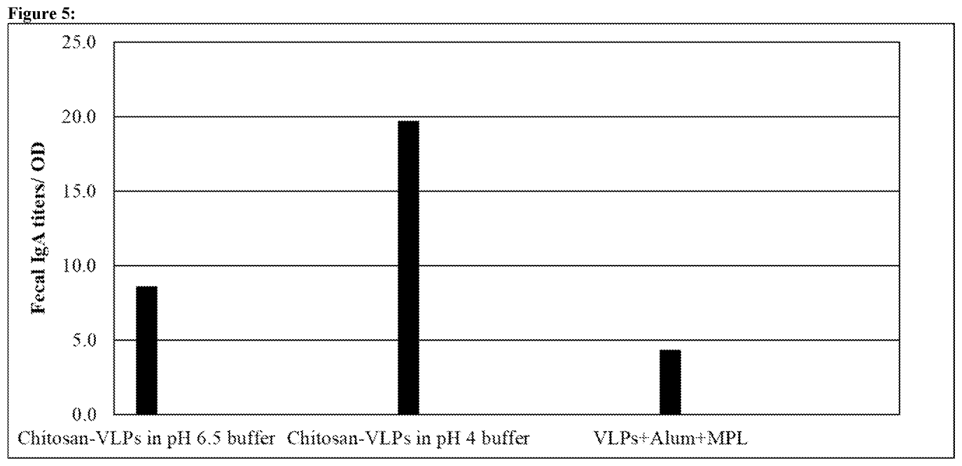

FIG. 6: The serum IgA titers in a murine model at different buffer concentrations corresponding to Example 6.

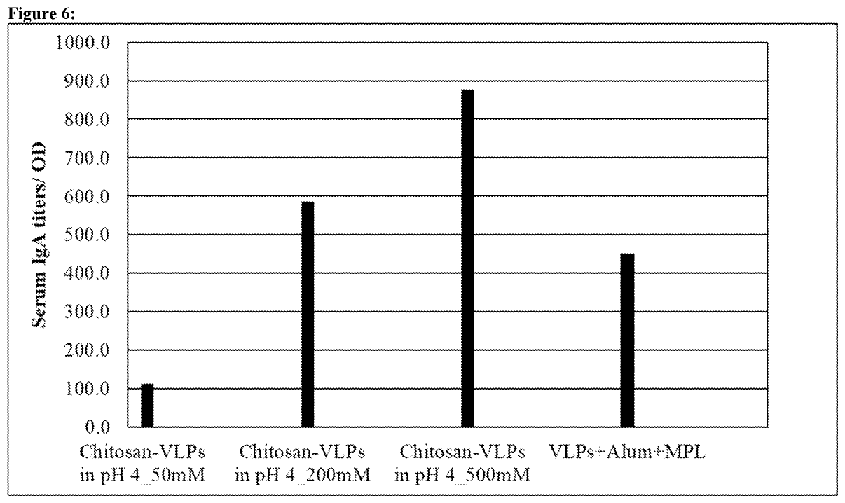

FIG. 7: The fecal IgA titers in a murine model at different buffer concentrations corresponding to Example 6.

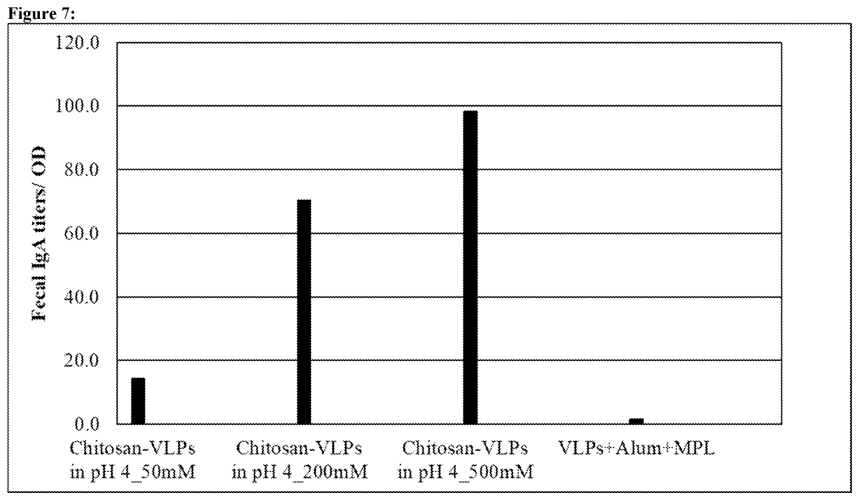

FIG. 8: The serum IgG titers in a murine model at different buffer concentrations corresponding to Example 6.

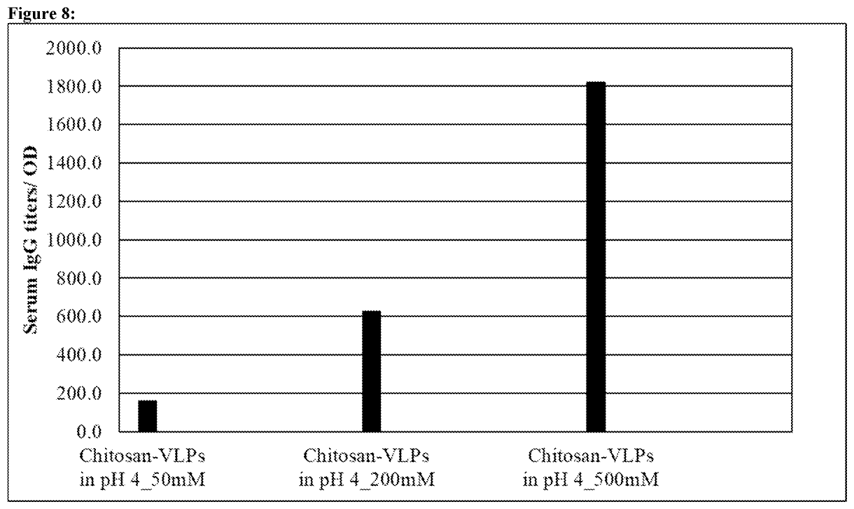

FIG. 9: The serum IgA titers in a murine model with different concentrations of chitosan to VLPs corresponding to Example 7.

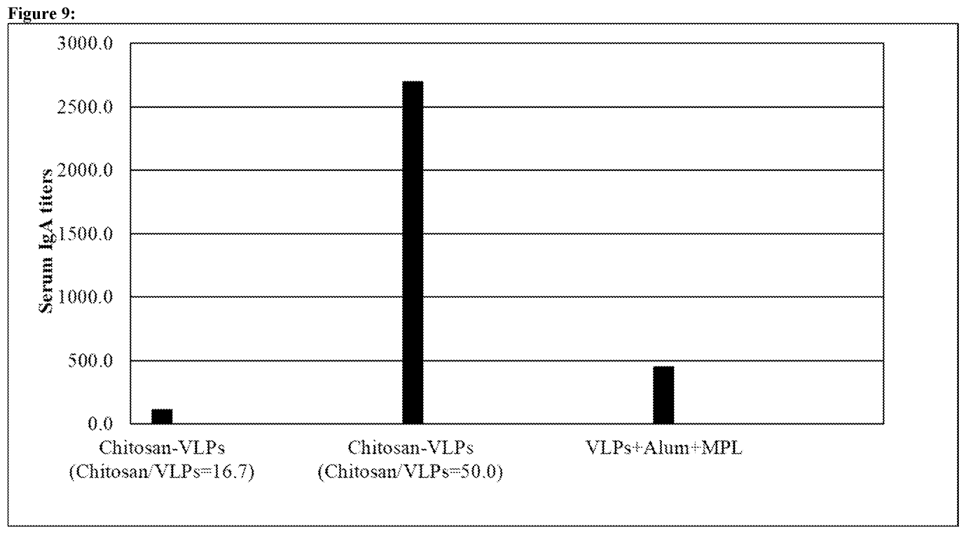

FIG. 10: The fecal IgA titers in a murine model with different concentrations of chitosan to VLPs corresponding to Example 7.

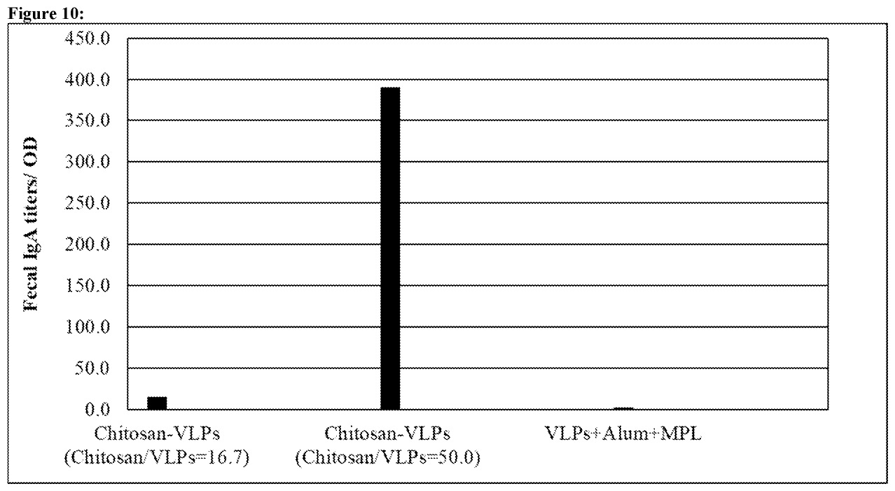

FIG. 11: The serum IgG titers in a murine model with different concentrations of chitosan to VLPs corresponding to Example 7.

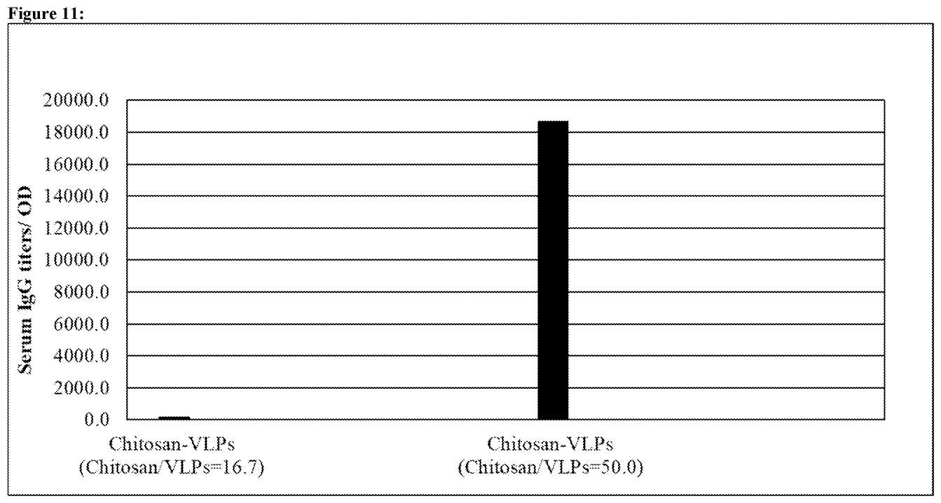

FIG. 12: The serum IgA titer in a murine model with naked and chitosan coated VLPs corresponding to Reference Example 1.

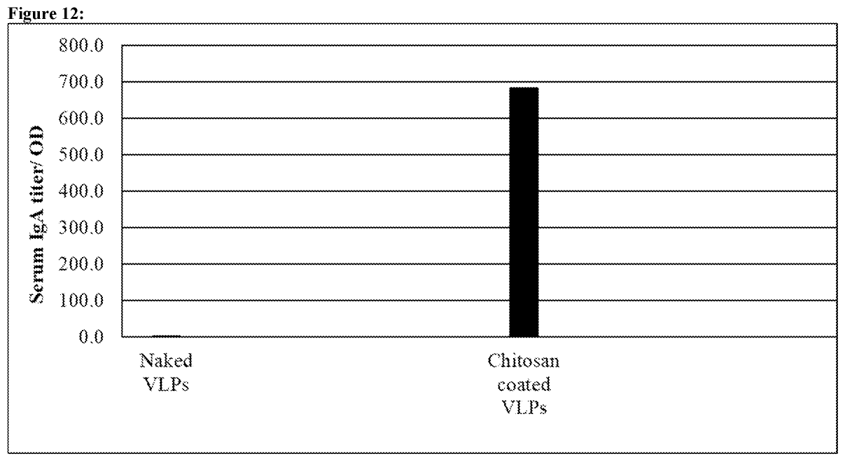

FIG. 13: The fecal IgA titer in a murine model with naked and chitosan coated VLPs corresponding to Reference Example 1.

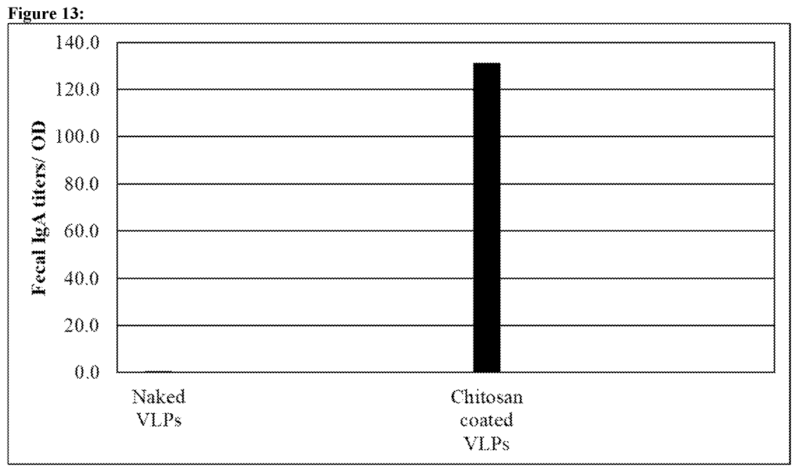

FIG. 14: The serum IgG titer in a murine model with naked and chitosan coated VLPs corresponding to Reference Example 1.

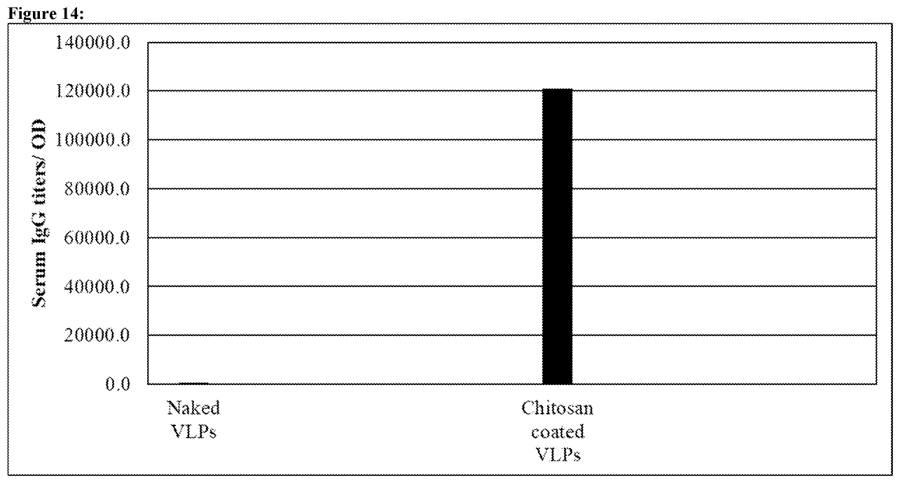

FIG. 15: The serum IgA titer in a murine model with chitosan coated VLPs administered to the jejunum or orally to four balb/c mice corresponding to Reference Example 2.

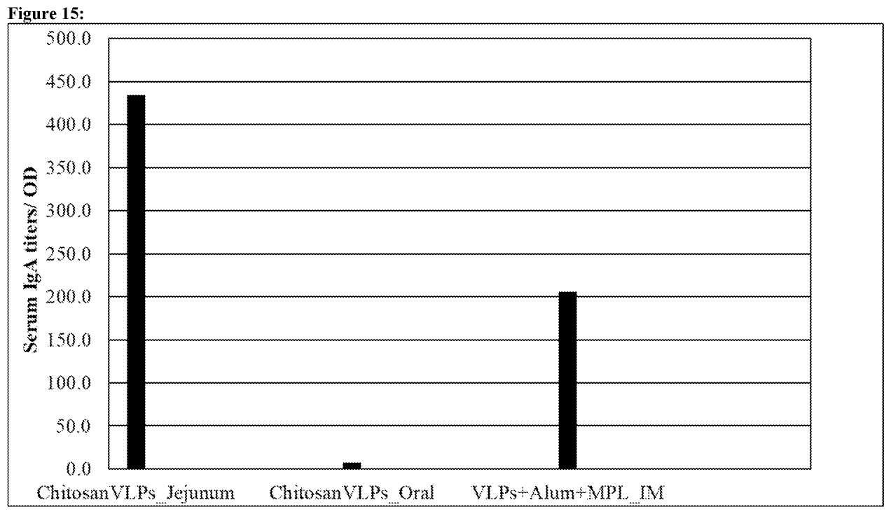

FIG. 16: The fecal IgA titer in a murine model with chitosan coated VLPs administered to the jejunum or orally to four balb/c mice corresponding to Reference Example 2.

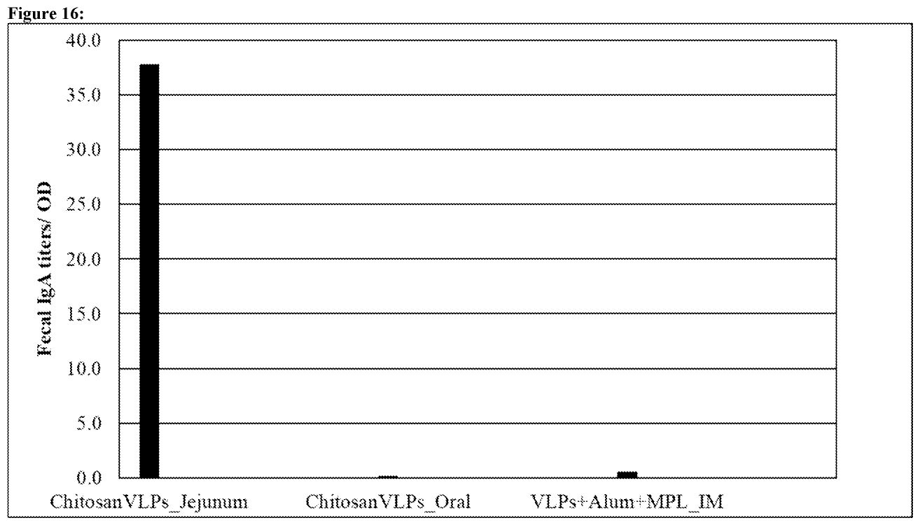

### DEFINITIONS

Unless defined otherwise, all technical and scientific terms used herein have the same meaning as commonly understood by one of ordinary skill in the art to which the invention pertains. Although, any methods and materials similar or equivalent to those described herein can be used in practice for testing of the present invention, the preferred materials and methods are described herein. In describing and claiming the present invention, the following terminology will be used in accordance with the definitions set out below.

Unless clearly indicated otherwise, use of the terms “a,” “an,” and the like refers to one or more.

The term “bioadhesion” is defined for purposes of the present invention to refer to the mechanism by which two biological materials are held together by interfacial forces.

The term “mucoadhesive” (or “mucosal adjuvant”) is defined for purposes of the present invention to refer to a material that exhibits bioadhesion and is thus used to bind a compound to a mucous membrane; this includes membranes at epithelial surfaces such as the gastrointestinal tract, the vagina, the lung, the eye etc. Without wishing to be bound by any theory, a mucoadhesive is any molecule which interacts with the mucous layer covering mucous membranes, and increases the residence time of the dosage form (such as e.g. VLPs) at the site of absorption, this may lead to an increased immune response. The mucoadhesive is preferably polymeric. According to the invention, the mucoadhesive contains one or more amino groups and is herein referred to as “amino group containing mucoadhesive.” A non-exclusive list of such mucoadhesives is provided in paragraph [0073]-[0080] below.

The term “amino group” within the meaning of this invention includes primary, secondary, tertiary and quaternary amine groups (RNH₂, R₂NH, R₃N, R₄N⁺).

The term “mucosal administration” refers to administration of a composition via a mucosal pathway, for example oral, intranasal, rectal or vaginal administration of a composition to a mammal.

The term “gastrointestinal barriers” is defined for purposes of the present invention to refer to barriers to the delivery of drug substances via an oral route such as the low pH of the stomach or the presence of protease enzymes in the digestive tract.

The term “derivatized” refers to the introduction of an amino group into a mucoadhesive.

The term “VLPs” is defined for purposes of the present invention to refer to virus like particles. Virus like particles, such as NV-VLPs are structurally similar to viruses, but lack the infectious genetic material and, therefore, are not infectious. VLPs are usually prepared by recombinant expression and spontaneous self-assembly of the capsid protein.

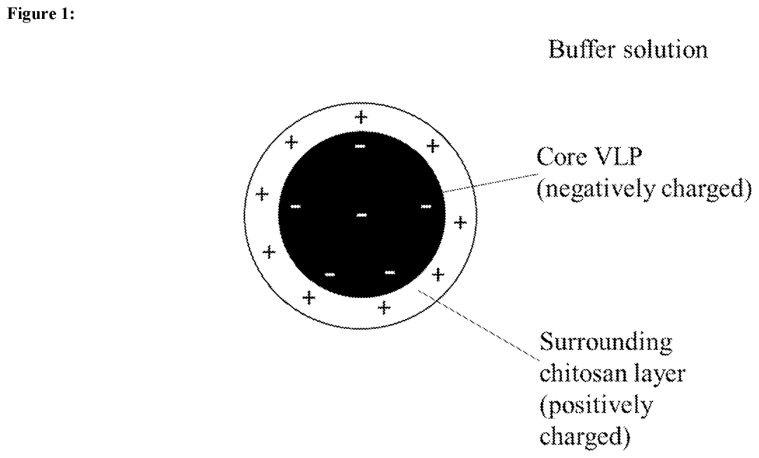

The terms “coated VLPs” or “VLPs with a surrounding mucoadhesive layer” are defined for purposes of the present invention to refer to VLPs, which have been suspended in a mucoadhesive solution according to the invention. Without wishing to be bound by any theory, it is believed that due to electrostatic attractions these VLPs have the mucoadhesive adsorbed on their surfaces. Reference is made to FIG. 1 for illustration purposes.

The term “naked VLPs” is defined for purposes of the present invention to refer to VLPs, which are not treated with a mucoadhesive and comparable materials, such as e.g. VLPs in water.

The term “viral infection” is defined for purposes of the present invention to refer to an infection caused by a virus or by multiple species or genotypes of virus.

The term “species” in accordance with the International Committee on Taxonomy of Viruses is defined as a monophyletic group of viruses whose properties can be distinguished from those of other species by multiple criteria. Norovirus is an example of a virus species.

The term “genotype” is defined for purposes of the present invention to refer to the genetic constitution of an organism, such as the Norovirus genotypes GI, GII, GIII and GIV.

The term “vaccine” is defined for purposes of the present invention to refer to a formulation, which contains VLPs of the present invention, in a form that is capable of being administered to a mammal and which elicits protective immunity to a viral infection in a mammal. Administering a vaccine to an animal is referred to as “vaccination.”

The term “adjuvant” is defined for purposes of the present invention to refer to a compound added to the aqueous composition in order to lead to an enhanced immune response when used as a mammalian vaccine. The adjuvant may for example be alum or MPL (Monophosphoryl lipid A).

The term “protective immunity” or “protective immune response” refers to immunity or eliciting an immune response against an infectious agent, which is exhibited by a mammal (e.g. a human), that prevents or ameliorates an infection or reduces at least one symptom thereof. Protective immunity can refer to an immune response in a healthy subject not experiencing the symptoms of a viral e.g. Norovirus infection, such as that generated via vaccination, or to an immune response elicited in a subject currently experiencing symptoms of a viral e.g. Norovirus infection. Specifically, induction of protective immune response from administration of the vaccine is evident by elimination or reduction in the duration or severity of the symptoms of the disease. A protective immune response that reduces or eliminates disease symptoms will reduce or stop the spread of a virus outbreak in a population. Clinical symptoms of e.g. gastroenteritis from Norovirus include nausea, diarrhea, loose stool, vomiting, fever and general malaise. A protective immune response against e.g. Norovirus results in a mammal not experiencing symptoms of gastroenteritis or experiencing a reduction in duration or severity of such symptoms.

The terms “IgA” and “IgG” refer to the antibodies Immunoglobulin A and Immunoglobulin G respectively within the meaning of this invention.

The term “Norovirus” is defined for purposes of the present invention to refer to members of the species Norovirus of the family Caliciviridae. In some embodiments, a Norovirus can include a group of related, positive-sense single-stranded RNA, non-enveloped viruses that can be infectious to human or non-human mammalian species. In some embodiments, Noroviruses can cause acute gastroenteritis in humans. Included within the group of Noroviruses are at least four genotypes (GI, GII, GIII, GIV) defined by nucleic acid and amino acid sequences, which comprise 15 genetic clusters. The major genotypes are GI and GII. Non-limiting examples of Noroviruses include Norwalk virus (NV, GenBank M87661, NP₀₅₆₈₂₁), Southampton virus (SHV, GenBank L07418), Desert Shield virus (DSV, U04469), Hesse virus (HSV), Chiba virus (CHV, GenBank AB042808), Hawaii virus (HV, GenBank U07611), Snow Mountain virus (SMV, GenBank U70059), Toronto virus (TV, Leite et al., Arch. Virol. 141:865-875), Bristol virus (BV), Jena virus (JV, AJ01099), Maryland virus (MV, AY032605), Seto virus (SV, GenBankAB031013), Camberwell (CV, AF145896), Lordsdale virus (LV, GenBank X86557), Grimsby virus (GrV, AJ004864), Mexico virus (MXV, GenBank U22498), Boxer (AF538679), C59 (AF435807), VA115 (AY038598), BUDS (AY660568), Houston virus (HoV), Minerva strain (EF 1269631), Laurens strain (EF 1269661), MOH (AF397156), Parris Island (PiV, AY652979), VA387 (AY038600), VA207 (AY038599), and Operation Iraqi Freedom (OIF, AY675554). Also included are Norovirus consensus VLPs, which are a construct representing a consensus sequence from two or more Norovirus strains such as GII.4 strains. Also included are Norovirus composite VLPs, which are derived from a consensus sequence from two or more Norovirus strains such that the consensus sequence contains at least one different amino acid as compared to each of the sequences of said two or more Norovirus strains. The construction of Norovirus composite VLPs is disclosed in WO 2010/017542, which is herewith incorporated by reference in its entirety.

The term “size average” is defined for purposes of the present invention to refer to the mean size of particles measured in a liquid using a Zetasizer Nano ZS (Malvern) as described in the Test Methods Section.

The term “comparison sample” is defined for purposes of the present invention to refer to a sample containing the same concentration of VLPs of the same genotype and tested under the same conditions as the test sample. The difference to the test sample is expressly mentioned.

The term “aqueous composition” is defined for purposes of the present invention to refer to a composition containing water, with up to 30% organic solvents dissolved within the composition, more preferably with up to 20%, or up to 10% organic solvents dissolved within the composition. Aqueous compositions may also comprise salts or other substances dissolved in them; as well as be mixtures or emulsions containing insoluble particles.

The terms “dry composition” or “composition in dry form” are defined for purposes of the present invention to refer to a solid material with a residual water content of less than about 10% w/w. Dried compositions are preferably dried to residual liquid (water) contents of 8% w/w or less, 5% w/w or less, or preferably from about 0.1% w/w to about 5% w/w, such as less than about 2% w/w, or less than about 1% w/w. Preferably, the residual water content is determined in accordance with the Karl Fisher Titration method.

Dry compositions may be prepared by any method known to a person skilled in the art. Common methods of drying pharmaceutical compositions include “spray drying” which involves spraying a liquid through a nozzle into a stream of gas, and “lyophilisation” which involves freezing the composition and removing the liquid using reduced pressure.

The term “serum” is defined for purposes of the present invention to refer to the blood serum. The term “serum titer” is defined for purposes of the present invention to refer to the concentration of antibody in the blood serum measured using ELISA as described in the Test Methods Section.

The terms “OD” and “OD_{450 nm}” are used interchangeably within the present invention and refer to the optical density of samples measured at 450 nm in a 96 well plate.

If not indicated otherwise “%” refers to “weight %”

The term “unit dosage form” refers to a dose of the composition suitable for a single therapeutic intervention in a mammal, such as in humans.

### DETAILED DESCRIPTION

### VLP Species and Production

The present invention relates to an aqueous composition comprising VLPs.

Virus-like particle(s) or VLPs refer to virus-like particle(s), produced from the capsid protein coding sequence of a virus, particularly a non-enveloped virus, and comprising antigenic characteristic(s) similar to those of the infectious virus.

In certain embodiments, the non-enveloped virus is selected from the group consisting of Calicivirus (e.g., a Norovirus or a Sapovirus), Picornavirus, Astrovirus, Adenovirus, Reovirus, Polyomavirus, Papillomavirus, Parvovirus, and Hepatitis E virus.

In certain embodiments, the aqueous composition may comprise VLPs derived from at least 1 species of virus, or VLPs derived from at least 2 different species of virus, or VLPs derived from at least 3 different species of virus, or VLPs derived from at least 4 different species of virus.

In certain embodiments, the aqueous composition may comprise VLPs selected from the group of species of Norovirus VLPs, Rotavirus VLPs, HPV VLPs, Influenza virus VLPs, dengue fever virus VLPs, hepatitis B virus VLPs. In certain preferred embodiments, the aqueous composition may comprise VLPs selected from the group of species of Norovirus VLPs and Rotavirus VLPs.

In certain embodiments, the composition may comprise VLPs of at least 1 genotype, or VLPs of at least 2 different genotypes, or VLPs of at least 3 different genotypes, or VLPs of at least 4 different genotypes.

In certain, such embodiments the composition may comprise one or more different genotypes of Norovirus VLP selected from the group consisting of Norovirus group 1 VLPs (GI), Norovirus group 2 VLPs (GII), Norovirus group 3 VLPs (GIII), and Norovirus group 4 VLPs (GIV). In certain, preferred embodiments the genotypes of the Norovirus VLPs may be selected from the group of Norovirus group 1 VLPs (GI) and Norovirus group 2 VLPs (GII). In certain more preferred embodiments, the genotypes of the Norovirus VLPs may be selected from the group of Norovirus group 1.1 VLPs (GI.1), Norovirus group 2.2 VLPs (GII.2) and Norovirus group 2.4 VLPs (GII.4), for example, Norwalk virus VLPs and a construct representing a consensus sequence or a composite sequence from several norovirus GII.4 strains. For example, the consensus sequence or composite sequence can be derived from Norovirus strains selected from the group consisting of Houston strain, Minerva strain and Laurens strain. In a particularly preferred embodiment, the Norovirus VLPs have a GII.4 composite amino acid sequence corresponding to SEQ ID NO: 1 of WO 2010/017542.

### Mucoadhesive

The mucoadhesive or mucosal adjuvant according to the present invention contains amino groups and is preferably polymeric. Without wishing to be bound by any theory, it is believed that the amino groups are present in protonated form in the aqueous composition to provide for a significant cationic charge. Without wishing to be bound by any theory, it is believed that the cationic mucoadhesive binds to the VLPs as a result of an electrostatic interaction between the VLPs and the mucoadhesive, see FIG. 1 for the purpose of illustration, producing VLPs which are coated with the mucoadhesive polymer. Furthermore, the charge of the positive mucoadhesive bound to the VLPs by electrostatic repulsion prevents aggregation of the VLPs.

The sufficient protonation of the amino groups and the cationic charge in the composition is achieved and maintained by the aqueous environment, which has a pH that is more acidic than the value of the pKa of the amino group on the mucoadhesive (i.e., the numeric value of the pH is smaller than the numeric value of the pKa). Consequently, it is preferred that the pH of the aqueous environment is adapted to the pKa of the amino group on the mucoadhesive. The mucoadhesive may, therefore, be admixed in the form of the base and the protonation of the amino group takes place in the composition due to the acidic environment; or the mucoadhesive may be admixed in the form of a salt with the amino group already protonated. The final degree of protonation will in any case be dependent on the pH of the aqueous composition. Alternatively, the amino group containing mucoadhesive in the aqueous composition according to the present invention may contain a quaternary ammonium group or quaternary ammonium groups, which are also positively charged.

The pKa refers to the pKa of the protonated form of the most basic amino group or the most basic amino groups in case of repeating units in a polymer of the mucoadhesive. The pKa as used herein refers to the pKa measured in an aqueous solution. The pKa is preferably determined using a potentiometric titration, it is normal for the pKa value to vary to a certain degree depending on the exact mathematical method, experimental setting and the exact titration curves used to calculate its value. Such parameters may change from material to material to be measured. Therefore, according to the invention the pKa refers to the higher value obtained from such measurement and/or method, in case variation is obtained. The pKa of the mucoadhesive if preferably from about 7.0 to 3.5, or from about 7.0 to 4.0, or from about 7.0 to 5.0. Chitosan is e.g. reported to exhibit a pKa of about 6.5, see e.g. Park et al. Bulletin of Korean Chemical Society 1983, 4, 68-72. According to the invention chitosan, in particular chitosan glutamate is combined with a pH of 5.5 and less. Thus, in a preferred embodiment of the invention the mucoadhesive is chitosan glutamate and the buffer has a pH of 5.5 or less, or 4.5 or less, or 4 or less, such as an acetate buffer, and the pH of the aqueous composition is 5.5 or less, or 4.5 or less, or 4 or less.

Additionally, it is contemplated that the buffer and the mucoadhesive advance the chemical and physicochemical stability of the composition containing VLPs against gastrointestinal barriers. Furthermore, the mucoadhesive prolongs the retention time of the composition in the intestines, which leads to increased absorption of the VLP containing composition. The composition, therefore, enhances the delivery of the VLPs to the target immune cells and consequently induces a desired immune response and/or a higher immune response than a composition without mucoadhesive.

Within the meaning of this invention, mucoadhesives are selected from molecules containing amino groups, in particular polymers. The amino group can be present in the molecule, such as a polymer, or subsequently introduced by derivatization.

In certain embodiments, the composition contains an amino group containing mucoadhesive selected from the group of polysaccharides (carbohydrate polymers), optionally derivatized polysaccharides (derivatized carbohydrate polymers), cross-linked derivatives of poly(acrylic acid), optionally derivatized polyvinyl alcohol, optionally derivatized polyvinyl pyrollidone, polypeptides, derivatized polypeptides, proteins, derivatized proteins, derivatized deoxyribonucleic acid, polyethylenimine and derivatives thereof.

In certain such embodiments, the mucoadhesive is a polysaccharide selected from the group of chitosan, chitosan salt, chitosan base, derivatized chitosan, aminodextran, dimetylaminoethyl dextran, glycosaminoglycans, derivatized glycosaminoglycans, cellulose derivatives, derivatized pectin, derivatized alginate, derivatized glycogen, derivatized amylose, derivatized amylopectin, derivatized chitin, derivatized stachyose, derivatized inulin, derivatized dextrin, derivatized dextran and other derivatized polysaccharides, such as mucin and other mucopolysaccharides.

Examples of glycosaminoglycans include, but are not limited to chondroitin sulfate, dermatan sulfate, keratan sulfate, heparin, heparan sulfate and hyaluronan.

Examples of cellulose derivatives include, but are not limited to derivatives of hydroxypropyl methylcellulose and carboxymethylcellulose.

Examples of polypeptides include, but are not limited to polylysine. Optionally the polypeptides are derivatized by the introduction of amino groups.

Examples of proteins include, but are not limited to lectin and fimbrial proteins. These proteins are if required derivatized by the introduction of amino groups.

In certain preferred embodiments, the mucoadhesive is chitosan, which may be admixed in the form of a chitosan base, or a chitosan salt. In certain such embodiments, the admixed mucoadhesive is selected from the group of chitosan glutamate, chitosan hydrochloride, chitosan acetate, chitosan lactate, chitosan alginate, chitosan ascorbate, trimethylated chitosan, methyl glycol chitosan, and carboxymethyl chitosan, in particular chitosan glutamate.

In certain such embodiments, the chitosan glutamate may have an average molecular weight of from 200 kDa to 600 kDa and/or a deacylation value of 75% to 90% and/or a viscosity of 20-200 mPa·s.

Examples of commercially available chitosan glutamate, which may be used in the present invention are given in the table below. For further information reference is also made to the product information provided by NovaMatrix as of July 2018.

### Commercially Available Types of Chitosan Glutamate from Various Chemical Suppliers

| | | Molecular | | |
|---|---|---|---|---|
| | Viscosity | Weight | Deacetylation | |
| Product Name | (mPa · s) | (kDa) | (%) | Supplier |
| PROTASAN | 20-200 | 200-600 | 75-90 | NovaMatrix, a |
| UP G 213¹ | | | | business unit of |
| | | | | FMC Health and |
| | | | | Nutrition |
| PROTASAN | >20 | <200 | 75-90 | NovaMatrix, a |
| UP G 113 | | | | business unit of |
| | | | | FMC Health and |
| | | | | Nutrition |
| PROTASAN | 20-200 | 200-600 | >90 | NovaMatrix, a |
| UP G 214 | | | | business unit of |
| | | | | FMC Health and |
| | | | | Nutrition |
| Chitosan | — | — | — | Heppe Medical |
| Glutamate | | | | Chitosan GmbH |
| Chitosan | — | — | — | CarboMer, Inc. |
| Glutamate | | | | |
| Chitosan | <200 | — | >90 | Golden-Shell |
| Glutamate | | | | Pharmaceutical Co. |
| | | | | Ltd. |

¹Chitosan glutamate brand used in the examples of the present invention.

In certain embodiments, the present invention is directed to an aqueous composition, wherein the concentration of admixed mucoadhesive is greater than about 8 mg/mL, or greater than about 10 mg/mL, or greater than about 12.5 mg/mL, or greater than about 15 mg/mL, or greater than about 30 mg/mL, or greater than about 50 mg/mL, preferably the maximum mucoadhesive concentration is about 100 mg/mL. The above numbers are based on the addition of chitosan glutamate. Equimolar amounts of other types of chitosan salt or chitosan base may also be used.

In certain such embodiments, the present invention is directed to an aqueous composition, wherein the concentration of admixed mucoadhesive is from about 8 mg/mL to about 50 mg/mL, or wherein the concentration of mucoadhesive is from about 10 mg/mL to about 30 mg/mL, or wherein the concentration of mucoadhesive is from about 10 mg/mL to about 20 mg/mL.

Often mucoadhesives with a higher concentration have a higher viscosity. This is for example the case for chitosan. Without wishing to be bound by any theory, it is assumed that this higher viscosity leads to higher gastrointestinal retention. Furthermore, it is believed that a higher concentration of chitosan also contributes to increasing the absorption of the composition and to inducing a desirable immune response, in addition to the properties of the chitosan itself.

### Composition

The present invention is directed to an aqueous composition comprising:
water,
virus like particles (VLPs),
at least one amino group containing mucoadhesive, and
at least one buffer optionally with a pH of less than about 6,
   wherein the virus like particles have a size average of no more than about 4 times the size average of a comparison sample of virus like particles without said mucoadhesive in water, and/or
   wherein the virus like particles have a size average of no more than about 400 nm.

In a certain aspect the aqueous composition of the paragraph above, wherein the at least one amino group containing mucoadhesive has a pKa of less than about 7, and wherein the pH of the aqueous composition is at least about 1 pH unit more acidic than the value of the pKa of the mucoadhesive.

In certain embodiments, the aqueous composition of the present invention has a pH of about 5.8 or less, or about 5.5 or less, or about 5.1 or less, or about 5 or less, or about 4.5 or less.

In certain embodiments, the aqueous composition of the present invention has a pH of at least about 0.7 pH units more acidic than the value of the pKa of the mucoadhesive, or has a pH of at least about 1 pH unit more acidic than the value of the pKa of the mucoadhesive, or has a pH of at least about 1.4 pH units more acidic than the value of the pKa of the mucoadhesive, or has a pH of at least about 1.5 pH units more acidic than the value of the pKa of the mucoadhesive, or has a pH of at least about 2 pH units more acidic than the value of the pKa of the mucoadhesive, or has a pH of at least about 2.5 pH units more acidic than the value of the pKa of the mucoadhesive, or has a pH of at least about 3 pH units more acidic than the value of the pKa of the mucoadhesive. As already mentioned, it is believed that at acidic pH values, which are at least 1 pH unit more acidic than the value of the pKa of the amino group on the mucoadhesive, the amino groups on the mucoadhesive will be significantly protonated. The mucoadhesive then binds to the VLPs through electrostatic interactions (see FIG. 1). The charged mucoadhesive surrounding the VLPs leads to electrostatic repulsion between the VLPs leading to reduced aggregation. This effect corresponds with an increased zeta potential of the VLPs. These highly charged VLPs are thought to repel one another leading to particles that are less than 4 times the size of the uncoated VLPs or less than about 400 nm.

In certain such embodiments, the present invention is directed to an aqueous composition, wherein the pH of the composition is from about 2.5 to about 5.5. Such compositions work well with mucoadhesives such as chitosan exhibiting a pKa of about 6.5.

In certain such embodiments, the pH of the aqueous composition is about 5 or less, or about 4.5 or less, or about 4 or less. In certain such embodiments, the pH of the aqueous composition ranges from about 4.5 to about 2.5, or from about 4.5 to about 3.5, such as, e.g., about pH 4 or about pH 3.

In certain embodiments, the pH of the aqueous composition is from about pH 4.0 to pH 4.4.

In order to stabilize the pH of the aqueous compositions a buffer is used, such as an acetate buffer. The pH of the buffer can be from about 2.5 to about 5.5, or about 3.0 to about 5.0 or about 3.5 to about 4.5.

Without wishing to be bound by any theory, it is believed that the buffer solution establishes the pH suitable for protonating the amino groups on the mucoadhesive and helps to improve the stability of the compositions against the low pH (pH 1-3) of the stomach. Furthermore, it is believed that the buffer helps to improve the stability of the composition in the intestinal tract by preventing activities of proteases in the intestine, which are high at intestinal pH (about pH 6-7).

In certain such embodiments, the present invention is directed to an aqueous composition, wherein the concentration of buffer is from about 25 mM to about 2000 mM, or from about 50 mM to about 1000 mM, or from about 50 mM to about 500 mM, or from about 200 mM to about 500 mM.

In certain embodiments, the present invention is directed to an aqueous composition, wherein the concentration of mucoadhesive is greater than about 8 mg/mL, or greater than about 10 mg/mL, or greater than about 12.5 mg/mL. In certain such embodiments the maximum mucoadhesive concentration is about 100 mg/mL, or about 50 mg/mL, or about 30 mg/mL. In certain such embodiments the mucoadhesive concentration ranges from about 8 mg/mL to about 100 mg/mL, or from about 10 mg/mL to about 50 mg/mL, or from about 12.5 mg/ml to about 30 mg/mL. The above numbers are based on the addition of chitosan glutamate, in case the mucoadhesive is chitosan. Equimolar amounts of other types of chitosan salt or chitosan base may also be used. In case of other mucoadhesives, the numbers should be interpreted as referring to the free base, wherein equimolar amounts of salts can be used likewise.

In certain embodiments, the present invention is directed to an aqueous composition, wherein the virus like particles have a size average of no more than about 4 times the size average of a comparison sample of virus like particles without said mucoadhesive in water, or no more than about 3 times the size average of a comparison sample of virus like particles without said mucoadhesive in water, or no more than about 2.5 times the size average of a comparison sample of virus like particles without said mucoadhesive in water.

In certain embodiments, the present invention is directed to an aqueous composition wherein, the virus like particles have a size average of no more than about 400 nm, such as from about 10 nm to about 350 nm, or from about 10 nm to about 300 nm, or from about 10 nm to about 250 nm or from about 10 nm to about 200 nm, or from about 10 nm to about 170 nm, or from about 10 nm to about 150 nm, or from about 10 nm to about 120 nm. Within the meaning of this invention, the size average of the virus like particles refers to the size average of the virus like particles in the composition according to the present invention. Without wishing to be bound by any theory, it is believed that these virus like particles are coated virus like particles (VLPs) or virus like particles with a surrounding mucoadhesive layer and that due to electrostatic attractions these virus like particles have the mucoadhesive adsorbed on their surfaces. The size average refers to the mean size of the virus like particles measured in a liquid using a Zetasizer Nano ZS (Malvern) as described in the Test Methods Section and refers to the mean size of the virus like particles including the surrounding mucoadhesive layer which may be present.

In certain embodiments, the present invention is directed to an aqueous composition, wherein the virus like particles have a positive zeta potential. In certain preferred embodiments, the virus like particles have a positive zeta potential of at least about 15 mV, or at least about 20 mV, or at least about 30 mV and optionally up to a maximum zeta potential of 100 mV. Within the meaning of this invention, the Zeta potential of the virus like particles refers to the Zeta potential of the virus like particles in the composition according to the present invention. Without wishing to be bound by any theory, it is believed that these virus like particles are coated virus like particles or virus like particles with a surrounding mucoadhesive layer and that due to electrostatic attractions these virus like particles have the mucoadhesive adsorbed on their surfaces. The Zeta potential refers to the Zeta potential of the virus like particles measured in a liquid using a Zetasizer Nano ZS (Malvern) as described in the Test Methods Section and refers to the Zeta potential of the virus like particles including the surrounding mucoadhesive layer which may be present.

In certain embodiments, the present invention is directed to an aqueous composition wherein the concentration of the virus like particles in the aqueous composition is at least about 50 μg/mL, or is at least about 100 μg/mL, or is at least about 250 μg/mL, or is at least about 500 μg/mL, or is at least about 600 μg/mL, or is at least about 700 μg/mL, or is at least about 800 μg/mL.

In certain embodiments, the present invention is directed to an aqueous composition wherein the weight ratio of mucoadhesive (such as chitosan glutamate) to VLPs is from about 0.5 to about 300, or from about 0.5 to about 100, or from about 10 to about 100, or from about 20 to about 100, or from about 30 to about 100, or from about 40 to about 100, or from about 10 to about 60, or from about 30 to about 60, or from about 40 to about 60, such as about 50. The above numbers are based on the addition of chitosan glutamate, in case of a chitosan mucoadhesive. Equimolar amounts of other types of chitosan salt or chitosan base may also be used. In case other mucoadhesives are used, the ratio is interpreted to refer to equimolar amounts.

In certain such embodiments, the weight ratio of admixed chitosan glutamate, or an equimolar amount of another type of chitosan salt or chitosan base, to VLPs is from about 30 to about 100, or from about 40 to about 60, preferably about 50.

In certain embodiments, the present invention is directed to an aqueous composition comprising:
water,
virus like particles (VLPs),
chitosan, and
at least one buffer with a pH of from about 2.5 to about 5;
wherein the virus like particles are Norovirus virus like particles;
   wherein the virus like particles have a size average of no more than about 400 nm, and
   wherein the weight ratio of admixed chitosan glutamate, or an equimolar amount of another type of chitosan salt or chitosan base, to VLPs is from about 30 to about 100, or from about 40 to about 60, preferably about 50.

### Dry Form

According to a further aspect of the invention, the composition is in the form of a dry composition. The dry composition has a residual liquid/water content of less than about 5% w/w, or less than about 4% w/w, or less than about 2% w/w, or less than about 1% w/w, or less than about 0.1% w/w obtainable by drying the aqueous composition described above. Such dry compositions may be obtained by drying the aqueous composition described above using a method known in the art. For example, dry compositions may be obtained by drying the aqueous composition using spray drying or an associated method or via lyophilisation. The dry composition can be stored and used for solid dosage forms, such as solid oral dosage forms.

### Method of Treatment/Medical Uses/Vaccination

In certain embodiments, the present invention is directed to a composition for use in a treatment for eliciting protective immunity to a viral infection in an animal, particularly in a mammal e.g. a human. In certain preferred embodiments, the present invention is directed to a composition for use in a mucosally administered treatment for eliciting protective immunity to a viral infection in a mammal. In certain more preferred embodiments, the present invention is directed to a composition for use in an orally administered treatment for eliciting protective immunity to a viral infection in a mammal.

In certain embodiments, the present invention is directed to a method of eliciting protective immunity to a viral infection in a mammal (e.g. a human) in need of such a treatment, comprising administering a composition comprising VLPs (as described above) to a mammal. In certain preferred embodiments, the present invention is directed to a method of eliciting protective immunity to a viral infection in a mammal in need of such a treatment, comprising mucosally administering a composition comprising VLPs (as described above) to a mammal. In certain more preferred embodiments, the present invention is directed to a method of eliciting protective immunity to a viral infection in a mammal in need of such a treatment comprising orally administering a composition comprising VLPs (as described above) to a mammal.

In certain embodiments, the present invention is directed to the use of a composition comprising VLPs (as described above) in the manufacture of a medicament for eliciting protective immunity to a viral infection in a mammal (e.g. a human). In certain preferred embodiments, the present invention is directed to the use of a composition comprising VLPs (as described above) in the manufacture of a mucosally administered medicament for eliciting protective immunity to a viral infection in a mammal. In certain more preferred embodiments, the present invention is directed to the use of a composition comprising VLPs (as described above) in the manufacture of an oral medicament for eliciting protective immunity to a viral infection in a mammal.

In certain embodiments, use of the composition according to the present invention in a treatment for eliciting protective immunity to a viral infection in a mammal (e.g. a human) leads to an elevated level of immunoglobin when administered to the mammal. For example, an elevated level of serum IgA, and/or an elevated level of serum IgG, and/or an elevated level of fecal IgA, and/or an elevated level of fecal IgG.

In certain such embodiments, use of the composition according to the present invention in a treatment for eliciting protective immunity to a viral infection in a mammal (e.g. a human) leads to an increase in the serum titer of Norovirus-specific functional antibodies compared to a mammal not receiving the treatment.

In certain embodiments, the method of eliciting protective immunity to a viral infection in a mammal (e.g. a human) comprising administering to a mammal in need of such treatment a composition according to the present invention, leads to an elevated level of immunoglobulin in the mammal. For example, an elevated level of serum IgA, and/or an elevated level of serum IgG, and/or an elevated level of fecal IgA, and/or an elevated level of fecal IgG.

In certain such embodiments, the method of eliciting protective immunity to a viral infection in a mammal (e.g. a human) comprising administering to a mammal in need of such treatment the composition according to the present invention, may lead to an increase in the serum titer of Norovirus-specific functional antibodies compared to a mammal not receiving the treatment.

In certain embodiments, the use of a composition according to the present invention in the manufacture of a medicament for eliciting protective immunity to a viral infection in a mammal (e.g. a human) leads to an elevated level of immunoglobulin when administered to the mammal. For example, an elevated level of serum IgA, and/or an elevated level of serum IgG, and/or an elevated level of fecal IgA, and/or an elevated level of fecal IgG.

In certain such embodiments, the use of a composition according to the present invention in the manufacture of a medicament for eliciting protective immunity to a viral infection in a mammal (e.g. a human) may lead to an increase in the serum titer of Norovirus-specific functional antibodies compared to a mammal not receiving the treatment.

In certain embodiments, the present invention is directed to a composition wherein an adjuvant is not included in the composition. Commonly used medical adjuvants include MPL (Monophosphoryl lipid A) and alum. Within this context alum is used to refer to a compound comprising aluminum hydroxide [Al(OH)₃], such as aluminum hydroxide gel. In certain embodiments, the composition when administered orally to a mammal without an adjuvant may lead to a comparable or elevated immune response compared to a composition containing an adjuvant administered via injection (e.g. intramuscularly). The immune response may be measured, for example, as an increase in serum IgA response, serum IgG or in fecal IgA response.

In certain embodiments, the composition of the present invention is provided as a unit dosage form.

### Process of Preparation

In certain embodiments, the present invention is directed to a process for preparing an aqueous composition comprising:
water,
virus like particles,
at least one amino group containing mucoadhesive, and
at least one buffer optionally with a pH of less than 6,
   wherein the virus like particles in the composition have a size average of no more than about 4 times the size average of a comparison sample of virus like particles without said mucoadhesive in water, and/or
   wherein the virus like particles have a size average of no more than about 400 nm, the process comprising at least the following steps:
(a) providing virus like particles,
(b) providing at least one amino group containing mucoadhesive in a buffered aqueous composition with at least one buffer optionally with a pH of less than about 6 to obtain a mucoadhesive containing aqueous composition,
(c) adding the virus like particles to the mucoadhesive containing aqueous composition.

In a certain aspect the process of the paragraph above wherein, the at least one amino group containing mucoadhesive has a pKa of less than about 7, and wherein the pH of the aqueous composition is at least about 1 pH unit more acidic than the value of the pKa of the mucoadhesive. In said certain aspect the process of the paragraph above wherein, step (b) involves providing at least one amino group containing mucoadhesive in a buffered aqueous composition with a pH of at least about 1 pH unit more acidic than the value of pKa of the mucoadhesive, to obtain a mucoadhesive containing aqueous composition.

In certain such embodiments, the present invention is directed to a process for preparing said aqueous composition, wherein the mucoadhesive added in step (b) is chitosan, such as chitosan base or chitosan salt. In certain preferred embodiments, the mucoadhesive is chitosan glutamate.

In certain embodiments, the present invention is directed to a product obtainable by the process described in paragraphs [0114] to [0116] above.

### EXAMPLES

The following Examples are included to demonstrate certain aspects and embodiments of the invention as described in the claims. It should be appreciated by those of skill in the art, however, that the following description is illustrative only and should not be taken in any way as a restriction of the invention.

### Materials Used in the Compositions of the Examples

| Materials | Manufacturer/Supplier |
|---|---|
| Chitosan glutamate | NovaMatrix, a business unit of FMC |
| (PROTASAN UP G 213)¹ | Health and Nutrition |
| Sodium acetate | Wako Pure Chemical Industries, |
| | Osaka, Japan |
| Acetic acid | Wako Pure Chemical Industries, |
| | Osaka, Japan |
| PBS (Phosphate buffered | Wako Pure Chemical Industries, |
| saline) buffer | Osaka, Japan |
| Citric acid | Wako Pure Chemical Industries, |
| | Osaka, Japan |
| Sodium citrate | Wako Pure Chemical Industries, |
| | Osaka, Japan |
| L-Histidine | Wako Pure Chemical Industries, |
| | Osaka, Japan |
| Alum | InvivoGen, CA, USA |
| MPL | Sigma-Aldrich, MO, USA |
| Balb/c mice | Charles River Laboratories Japan., |
| | Yokohama, Japan |
| Needles (Gauge 27G and 30G) | Terumo Corporation, Tokyo, Japan |
| Goat Anti-Mouse IgG-HRP | Southern Biotech, AL, USA |
| Rat Anti-Mouse IgA HRP | Southern Biotech, AL, USA |
| One-step ABTS | Kirkegaard & Perry Laboratories, |
| | MD, USA |
| 1-step Ultra TMB-ELISA | Thermo Fisher Scientific, MA, USA |
| Tween-20 | Bio-Rad, CA, USA |
| Protease Inhibitor Cocktail | Wako Pure Chemical Industries, |
| Osaka, Japan | |

¹Chitosan glutamate Protosan UP G 213 has a viscosity of 20-200 mPa.sm a molecular weight of 200-600 kDa, and is 75-90% deacetylated.

### Virus Like Particles (VLPs) Used as Norovirus VLPs

In the Examples section virus like particles are referred to using the following abbreviations:

| VLP Name | Abbreviation |
|---|---|
| Norwalk virus VLPs¹ | GI.1 VLPs |
| Consensus VLPs² | GII.4 VLPs |

¹Norwalk virus (NV, GenBank M87661, NP056821)
²Consensus VLPs having the sequence of SEQ ID NO: 1 of WO 2010/017542

### Test Methods

### Measurement of Average Particle Size (Size Average), Polydispersity Index and Zeta Potential

The average particle size (size average), polydispersity index (PDI) and zeta potential were measured using a Zetasizer Nano ZS (Malvern) after diluting 10 times with the buffer used in the sample.

The size average (Zeta potential and PDI) was measured at 25° C. in disposable polystyrene cuvettes. A sample containing 1 mL of test solution was loaded into each cuvette. Measurement was conducted no later than 10 minutes after preparing the sample.

The refractive index of the cuvette material was set as 1.590 and the viscosity of the dispersant was set as 0.8872 cP. The measurement angle was set as 173 degrees backscatter.

### ELISA (Enzyme Linked Immunosorbent Assay)

ELISA was performed as follows: Sandwich ELISAs utilizing 96 well plates coated with VLPs at a concentration of 5 μg/mL in PBS were performed to determine specific anti-VLP IgA and specific anti-VLP IgG responses for each specimen. Anti-mouse IgA antibody reagents and anti-mouse IgG antibody reagents were used as secondary antibodies. One-step Ultra TMB-ELISA was used as the substrate for the IgA assay and One-step ABTS was used as substrate for the IgG assay. The titers were defined as the highest dilution to reach an OD (Optical density) of 0.5 at 450 nm.

### Serum IgA and Serum IgG

Approximately 1 mL of blood was collected from each mouse under anesthesia before the mouse was euthanized. This was centrifuged to obtain serum and then added to an ELISA plate as described above to monitor the immune response.

### Fecal IgA

Approximately 200 mg of feces was collected from each mouse cage every two hours. This was then homogenized after adding extraction buffer (PBS, 0.1% Tween-20, 1/10 Volume Protease Inhibitor Cocktail), centrifuged at 15,000 rpm at 4° C., and then the supernatant was added to an ELISA plate as described above.

### Example 1 (In Vitro Tests)

### Preparation of VLP Solutions

Norovirus VLPs were prepared as follows: Synthetic gene construct for composite sequence for capsid domains for one of the different groups of Norovirus was cloned into recombinant baculovirus. Infection of Sf9 insect cells demonstrated high yield of production of VLP. A 40 mL aliquot of a P2 pFastBac recombinant baculovirus stock for the composite VLP VP 1 gene was processed with a sucrose gradient to verify the expression and assembly of composite VLPs. The conditioned media was first layered onto a 30% sucrose cushion and then centrifuged at 140 K×g to pellet the VLP. The pellet was resuspended, layered onto a sucrose gradient and then centrifuged at 140 K×g. A visible white layer was observed within the gradient after centrifugation. 500 μL fractions from the gradient were collected and then analyzed by SDS-PAGE I Coomassie gel. The expected banding pattern for composite VLP at −56 kDa was observed within the sucrose gradient fractions. Using a high pressure liquid chromatography system with a running buffer of 20 mM Tris 150 mM NaCl pH 7 at a flow rate of 0.5 mL/minute, a 50 μL aliquot of the composite expression cell culture supernatant was loaded on to a Superose-6 size exclusion column. An intact VLP peak was observed at ˜15.3 minutes at 280 nm and 220 nm confirming integrity of the composite VLPs. Composite VLPs were also purified from conditioned media that had been filtered prior to using column chromatography. Conditioned media was processed by cation exchange chromatography. The cation exchange elution fraction was further purified by hydrophobic interaction chromatography (HIC). The HIC elution fraction was concentrated and buffer exchanged by tangential flow filtration. The final product was sterile filtered and stored at 4° C. 500 ng of the purified composite VLPs was analyzed by silver-stained SDS-PAGE. Using a high pressure liquid chromatography system with a running buffer of 20 mM Tris 150 mM NaCl pH 7 at a flow rate of 1.0 mL/minute, a 50 μL aliquot of the purified CM3 composite VLPs was loaded on to a Superose-6 size exclusion column. An intact VLP peak was observed at ˜7.5 minutes at 280 nm confirming integrity of the composite VLPs.

Chitosan solutions were prepared by dissolving 53.36 mg of chitosan glutamate in 3.34 mL of buffer to give a solution with a concentration of 15.98 mg/mL of chitosan glutamate, as specified in Table 1.

The chitosan solutions were then used to prepare chitosan coated VLPs by following the steps below:
a container was filled with a 1054, aliquot of the chitosan solution,
50 μg of GI VLPs in 20 mM histidine buffer pH 6.5 containing 1 mM NaCl (11 μL) and 50 μg of GII VLPs in 20 mM histidine buffer pH 6.8 containing 200 mM NaCl and 20% sucrose (9 μL) were added,
the resulting solution was then mixed.

Mixing of samples was carried out using a vortex mixer at speed of from about 100 to about 3,200 rpm, for a period of from about 1 second to about 20 seconds.

This led to a ratio of chitosan to VLPs in the formulation of 16.7.

Naked VLPs were prepared by simply combining 50 μg of both GI VLPs (11 μL) and GII VLPs (9 μL) and diluting with a 1054, aliquot of buffer as specified in Table 1.

The results of this test are shown in Table 1.

TABLE 1
Particle size, polydispersity index (PDI) and zeta potential of
VLPs at different pHs

| | | | Average | | |
|---|---|---|---|---|---|
| | | | (mean) | | Zeta |
| | | Final pH in | particle size | | potential |
| | Buffer | composition | (nm) | PDI | (mV) |
| Chitosan | 50 mM acetate | 4.2 | 72.3 | 0.383 | 50.2 |
| coated | buffer (pH 3) | | | | |
| VLPs | 50 mM acetate | 4.4 | 66.4 | 0.425 | 40.3 |
| | buffer (pH 4) | | | | |
| | 50 mM acetate | 5.1 | 99.6 | 0.553 | 35.9 |
| | buffer (pH 5) | | | | |
| | 20 mM histidine | 5.8 | 568.5 | 0.530 | 18.8 |
| | buffer (pH 6.5) | | | | |
| | 10 mM PBS | n.d. | 846.0 | 1.000 | 25.1 |
| | (pH 7.4) | | | | |
| Naked | 50 mM acetate | 3.8 | 53.9 | 0.315 | n.d. |
| VLPs | buffer (pH 3) | | | | |
| | 50 mM acetate | 4.2 | 60.4 | 0.391 | n.d. |
| | buffer (pH 4) | | | | |
| | 50 mM acetate | 5.1 | 83.3 | 0.548 | n.d. |
| | buffer (pH 5) | | | | |
| | 20 mM histidine | 6.5 | 50.4 | 0.242 | −16.2 |
| | buffer (pH 6.5) | | | | |

n.d.: not determined

### Example 2 (In Vitro Tests)

Chitosan solutions were prepared as in Example 1 using five different concentrations of pH 4 acetate buffer. These solutions were then used to produce chitosan coated VLPs as described in Example 1.

The results of these tests are shown in Table 2.

TABLE 2
Particle size, polydispersity index and zeta potential of VLPs in
different buffer strengths

| | | Average | | |
|---|---|---|---|---|
| | Final pH | (mean) | | Zeta |
| | in the | particle | | potential |
| Buffer | composition | size (nm) | PDI | (mV) |
| 50 mM acetate buffer (pH 4) | 4.4 | 66.4 | 0.425 | 40.3 |
| 100 mM acetate buffer (pH 4) | n.d. | 67.0 | 0.424 | 36.9 |
| 150 mM acetate buffer (pH 4) | n.d. | 69.0 | 0.420 | 32.1 |
| 200 mM acetate buffer (pH 4) | n.d. | 87.8 | 0.216 | 29.8 |
| 500 mM acetate buffer (pH 4) | 4.0 | 90.7 | 0.492 | 17.9 |

n.d.: not determined

### Example 3 (In Vitro Tests)

Chitosan solution, with a concentration of 15.98 mg/mL, was prepared as in Example 1 in 50 mM acetate buffer pH 4.

Chitosan coated VLPs were prepared as in Example 1 with 50 μg of GI VLPs and 50 μg of GII VLPs; however, the amount of chitosan solution added was varied, in order to vary the ratio of chitosan to VLPs in each formulation. The exact amount of chitosan solution added is shown in Table 3.

The particle size, PDI and zeta potential were then measured, as described in the test methods section.

The results of this test are shown in Table 3.

TABLE 3
Particle size, polydispersity index and zeta potential of VLPs with
different ratios of chitosan solution added.

| Amount of | | | | |
|---|---|---|---|---|
| chitosan | Ratio of | Average (mean) | | Zeta |
| solution | chitosan to | particle size | | potential |
| added (μL) | VLP | (nm) | PDI | (mV) |
| 3 | 0.5 | 50.0 | 0.305 | 29.9 |
| 16 | 2.5 | 51.8 | 0.308 | 27.7 |
| 105 | 16.7 | 66.4 | 0.425 | 40.3 |
| 210 | 33.5 | 83.5 | 0.390 | 39.0 |
| 314 | 50.0 | 96.0 | 0.455 | 38.2 |

### Example 4 (In Vivo Tests)

### Preparation of Drug Solution

Chitosan solutions were prepared in a similar manner to that described for Example 1 above, using 20 mM histidine buffer (pH 6.5) and 50 mM acetate buffer (pH 4) respectively. A 26.68 mg aliquot of chitosan glutamate was dissolved in 1.673 mL of each buffer solution, to give two different chitosan solutions, one at pH 4 and one at pH 6.5.

The chitosan solutions were then used to prepare chitosan coated VLPs by following the steps below:
a container was filled with a 1.673 mL aliquot of chitosan solution,
800 μg of GI VLPs (180 μL) and 800 μg of GII VLPs (147 μL) respectively were added,
the resulting solution, with a total volume of 2 mL was then mixed using a vortex mixer.

This led to a ratio of chitosan to VLPs in each solution of 16.7.

### Preparation of the Positive Control

Positive control drug solutions were prepared by following the steps below:
(1) a container was filled with 600 μg of GI VLPs and 600 μg of GII VLPs respectively,
(2) a 15 mg aliquot of aluminum hydroxide gel was added to the container,
(3) a 1.5 mg aliquot of monophosphoryl lipid A (MPL) was added to the container,
(4) 20 mM histidine buffer was added to make the final volume of the positive control up to 1.5 mL,
(5) the mixture was mixed using a vortex mixer.

### Animal Testing

A 250 μL aliquot of each formulation was subsequently administered to female balb/c mice (n=4 per group) on day 0 and day 22. Drug substance was administered orally and the positive control was administered intramuscularly.

On day 29 of the study serum and feces samples were collected. GI VLP-specific Immunoglobulin A (IgA) in the serum and feces was measured using ELISA (Enzyme Linked Immunosorbent Assay).

The results of this study are shown in table 4.

TABLE 4
Serum IgA and fecal IgA titer for samples tested in mice

| | | | GI | GII | Chitosan | | | | Serum IgA titer | | | Fecal IgA titer | | |
|---|---|---|---|---|---|---|---|---|---|---|---|---|---|---|
| | | Admin | VLPs | VLPs | glutamate | Alum | MPL | Vol. | (OD_{450 nm}) | | | (OD_{450 nm}) | | |
| | Buffer | Route | (μg)¹ | (μg)¹ | (μg)¹ | (μg)¹ | (μg)¹ | (μL) | Mean | Min | Max | Mean | Min | Max |
| Drug | 20 mM | Oral | 100 | 100 | 3335 | — | — | 250 | 54.4 | 8.5 | 129.6 | 8.6 | 1.5 | 23.5 |
| substance | Histidine | | | | | | | | | | | | | |
| (pH 6.5) | (pH 6.5) | | | | | | | | | | | | | |
| Drug | 50 mM | Oral | 100 | 100 | 3335 | — | — | 250 | 273.7 | 112.5 | 593.2 | 19.7 | 4.0 | 60.5 |
| substance | Acetate | | | | | | | | | | | | | |
| (pH 4) | (pH 4) | | | | | | | | | | | | | |
| Positive | 20 mM | Intra- | 100 | 100 | — | 250 | 2500 | 250 | 220.3 | 113.1 | 385.1 | 4.3 | 1.3 | 5.9 |
| control | Histidine | muscular | | | | | | | | | | | | |
| | (pH 6.5) | | | | | | | | | | | | | |

¹Total amount contained in the sample injected into the mouse. All results were obtained using 4 mice in total.

### Example 5 (In Vivo Tests)

### Preparation of Drug Solution

Chitosan solution was prepared in a similar manner to that described for Example 1 above, using acetate buffer (pH 4) of three different concentrations (50 mM, 200 mM and 500 mM). For each solution, a 26.68 mg aliquot of chitosan glutamate was dissolved in 1.673 mL of acetate buffer, giving three different solutions of different acetate buffer concentrations.

The chitosan solutions were then used to prepare chitosan coated VLPs by following the steps below:
(1) a container was filled with a 1.673 mL aliquot of chitosan solution,
(2) 800 μg of GI VLPs (180 μL) and 800 μg of GII VLPs (147 μL) respectively were added to the container,
(3) the resulting solution, with a total volume of 2 mL, was then mixed using a vortex mixer.

This led to a ratio of chitosan to VLPs in the formulation of 16.7.

### Preparation of Positive Control

Positive control drug solutions were prepared by following the steps below:
(1) a container was filled with 1000 μg of GI VLPs and 1000 μg of GII VLPs respectively,
(2) a 25 mg aliquot of aluminum hydroxide gel (alum) was added to the container,
(3) a 2.5 mg aliquot of monophosphoryl lipid A (MPL) was added to the container,
(4) 20 mM histidine buffer (pH 6.5) was added to make the final volume up to 2.5 mL,
(5) the mixture was mixed using a vortex mixer.

### Animal Testing

A 250 μL aliquot of each formulation was subsequently administered to female balb/c mice (n=4) on day 0 and day 22 (each formulation is summarized in Table 5). The drug substance was administered orally and the positive control was administered intramuscularly.

On day 29 of the study serum and feces samples were collected. GI VLP-specific fecal Immunoglobulin A (IgA) and VLP-specific Immunoglobulin G (IgG) in the serum were measured using ELISA (Enzyme Linked Immunosorbent Assay).

The results of this study are shown in table 5.

TABLE 5
Serum IgA, fecal IgA and serum IgG titer for samples tested in mice in Example 5

| | | | GI | GII | Chitosan | | | | Serum IgA titer | | | Fecal IgA titer | | | Serum IgG titer | | |
|---|---|---|---|---|---|---|---|---|---|---|---|---|---|---|---|---|---|
| | | Admin. | VLPs | VLPs | glutamate | Alum | MPL | Vol. | (OD_{450 nm}) | | | (OD_{450 nm}) | | | (OD_{450 nm}) | | |
| | Buffer | Route | (μg) | (μg) | (μg) | (μg) | (μg) | (μL) | Mean | Min | Max | Mean | Min | Max | Mean | Min | Max |
| Drug | 50 mM | Oral | 100 | 100 | 3335 | — | — | 250 | 112.7 | 18.8 | 208.1 | 14.4 | 1.3 | 34.1 | 159.3 | 15.2 | 322.6 |
| substance | acetate | | | | | | | | | | | | | | | | |
| (50 mM | buffer | | | | | | | | | | | | | | | | |
| buffer) | (pH 4) | | | | | | | | | | | | | | | | |
| Drug | 200 mM | Oral | 100 | 100 | 3335 | — | — | 250 | 586.2 | 231.4 | 1004 | 70.3 | 12.1 | 133.1 | 625.3 | 519.2 | 760.0 |
| substance | acetate | | | | | | | | | | | | | | | | |
| (200 mM | buffer | | | | | | | | | | | | | | | | |
| buffer) | (pH 4) | | | | | | | | | | | | | | | | |
| Drug | 500 mM | Oral | 100 | 100 | 3335 | — | — | 250 | 876.9 | 144.4 | 2961 | 98.3 | 19.2 | 312.2 | 1819 | 90.9 | 6820 |
| substance | acetate | | | | | | | | | | | | | | | | |
| (500 mM | buffer | | | | | | | | | | | | | | | | |
| buffer) | (pH 4) | | | | | | | | | | | | | | | | |
| Positive | 20 mM | Intra- | 100 | 100 | — | 250 | 2500 | 250 | 449.1 | 0.0 | 769.2 | 1.6 | 0.0 | 3.1 | NA | NA | NA |
| control | histidine | muscular | | | | | | | | | | | | | | | |
| | buffer | | | | | | | | | | | | | | | | |
| | (pH 6.5) | | | | | | | | | | | | | | | | |

All results were obtained using 4 mice in total.

### Example 6 (In Vivo Tests)

### Preparation of Drug Solution

Chitosan solutions were prepared in a similar manner to that described for Example 1 above, using 50 mM acetate buffer (pH 4). Solutions were prepared by dissolving 15.95 mg of chitosan glutamate in 1 mL of buffer.

The chitosan solutions were then used to prepare chitosan coated VLPs by following the steps below:
(1) a container was filled with an aliquot of 1.673 mL of chitosan solution,
(2) 800 μg of GI VLPs (180 μL) and 800 μg of GII VLPs (147 μL) respectively were then added,
(3) the resulting solution, with a total volume of 2 mL, was then mixed using a vortex mixer.

This led to a ratio of chitosan to VLPs in the composition of 16.7.

A further solution was prepared by following the steps below:
(1) a container was filled with an aliquot of 5.673 mL of chitosan solution,
(2) 800 μg of GI VLPs (180 μL) and 800 μg of GII VLPs (147 μL) respectively were then added,
(3) the resulting solution, with a total volume of 6 mL, was then mixed using a vortex mixer.

This led to a ratio of chitosan to VLPs in the composition of 50.

### Preparation of Positive Control

Positive control drug solutions were prepared by following the steps below:
(1) a container was filled with 1000 μg of GI VLPs and 1000 μg of GII VLPs respectively,
(2) a 25 mg aliquot of aluminum hydroxide gel (alum) was added to the solution,
(3) a 2.5 mg aliquot of monophosphoryl lipid A (MPL) was added to the solution,
(4) an aliquot of 20 mM histidine buffer (pH 6.5) was added to make the final volume up to 2.5 mL,
(5) the solution was mixed using a vortex mixer.

### Animal Testing

A 250 μL or 750 μL aliquot of each formulation was subsequently administered to female balb/c mice (n=4) on day 0 and day 22, with 250 μL of control substance being used. The drug substance was administered orally and the positive control was administered intramuscularly.

On day 29 of the study serum and feces samples were collected. GI VLP-specific serum and fecal Immunoglobulin A (IgA) and VLP-specific Immunoglobulin G (IgG) in serum were measured using ELISA (Enzyme Linked Immunosorbent Assay), as described above.

The results of this study are shown in Table 6.

TABLE 6
Serum IgA, fecal IgA and serum IgG titer for samples tested in mice in Example 6

| | | | GI | GII | Chitosan | | | | Serum IgA titer | | | Fecal IgA titer | | | Serum IgG titer | | |
|---|---|---|---|---|---|---|---|---|---|---|---|---|---|---|---|---|---|
| | | Admin. | VLPs | VLPs | glutamate | Alum | MPL | Vol. | (OD_{450 nm}) | | | (OD_{450 nm}) | | | (OD_{450 nm}) | | |
| | Buffer | Route | (μg) | (μg) | (μg) | (μg) | (μg) | (μL) | Mean | Min | Max | Mean | Min | Max | Mean | Min | Max |
| Chitosan- | 50 mM | Oral | 100 | 100 | 3335 | — | — | 250¹ | 112.7 | 18.8 | 208.1 | 14.4 | 1.3 | 34.1 | 159.3 | 15.2 | 322.6 |
| VLPs | acetate | | | | | | | | | | | | | | | | |
| (Chitosan/ | buffer | | | | | | | | | | | | | | | | |
| VLPs = | (pH 4) | | | | | | | | | | | | | | | | |
| 16.7) | | | | | | | | | | | | | | | | | |
| Chitosan- | 50 mM | Oral | 100 | 100 | 10005 | — | — | 750¹ | 2700 | 2002 | 3150 | 389.2 | 183.9 | 574.1 | 18660 | 4603 | 32500 |
| VLPs | acetate | | | | | | | | | | | | | | | | |
| (Chitosan/ | buffer | | | | | | | | | | | | | | | | |
| VLPs = | (pH 4) | | | | | | | | | | | | | | | | |
| 50.0) | | | | | | | | | | | | | | | | | |
| VLPs + | 20 mM | Intra- | 100 | 100 | — | 250 | 2500 | 250 | 449.1 | 0.0 | 769.2 | 1.6 | 0.0 | 3.1 | NA | NA | NA |
| Alum + | histidine | muscular | | | | | | | | | | | | | | | |
| MPL | buffer | | | | | | | | | | | | | | | | |
| | (pH 6.5) | | | | | | | | | | | | | | | | |

¹The same total mass of VLPs was administered in each case.

### Reference Example 1

### Preparation of Drug Solution

Chitosan solution was prepared in a similar manner to that described for Example 1 above, using 20 mM histidine buffer (pH 6.5). A 13.34 mg aliquot of chitosan glutamate was dissolved in 0.837 mL of buffer to prepare a chitosan solution.

The chitosan solutions were then used to prepare chitosan coated VLPs by following the steps below:
(1) a container was filled with aliquots of 0.837 mL of chitosan solution,
(2) 400 μg of GI VLPs (90 μL) and 400 μg (73 μL) of GII VLPs respectively were also added,
(3) the resulting solution of total volume 1 mL was then mixed using a vortex mixer.
   This led to a ratio of chitosan to VLPs in the formulation of 16.7.

### Preparation of Negative Control

For the negative control naked VLPs were prepared in 20 mM histidine buffer (pH 6.5):
(1) a container was filled with 400 μg of GI VLPs (90 μL) and 400 μg of GII VLPs (73 μL) respectively,
(2) 20 mM histidine buffer (pH 6.5) was added to make the final volume up to 1 mL in each case,
(3) the solution was then mixed using a vortex mixer.

### Animal Testing

A 50 μL aliquot of each formulation was directly administered to the jejunum of female balb/c mice (n=4) on day 0 and day 22.

On day 29 of the study serum and feces samples were collected. GI VLP-specific serum and fecal Immunoglobulin A (IgA) and VLP-specific Immunoglobulin G (IgG) in serum were measured using ELISA (Enzyme Linked Immunosorbent Assay).

TABLE 7
Serum IgA, fecal IgA and serum IgG titer for samples tested in mice in Reference Example 1

| | | | GI | GII | Chitosan | | Serum IgA titer | | | Fecal IgA titer | | | Serum IgG titer | | |
|---|---|---|---|---|---|---|---|---|---|---|---|---|---|---|---|
| | | Admin | VLPs | VLPs | glutamate | Vol. | (OD_{450 nm}) | | | (OD_{450 nm}) | | | (OD_{450 nm}) | | |
| | Buffer | Route | (μg) | (μg) | (μg) | (μL) | Mean | Min | Max | Mean | Min | Max | Mean | Min | Max |
| Negative | 20 mM | Intra- | 20 | 20 | — | 50 | 1.2 | 0.0 | 3.6 | 0.4 | 0.0 | 0.8 | 134.1 | 67.4 | 167.4 |
| control | Histidine | jejunum | | | | | | | | | | | | | |
| | buffer | | | | | | | | | | | | | | |
| | (pH 6.5) | | | | | | | | | | | | | | |
| Chitosan | 20 mM | Intra- | 20 | 20 | 667 | 50 | 682.0 | 11.3 | 1614 | 131.0 | 15.6 | 354.4 | 120530 | 1123 | 297600 |
| coated | Histidine | jejunum | | | | | | | | | | | | | |
| VLPs | buffer | | | | | | | | | | | | | | |
| | (pH 6.5) | | | | | | | | | | | | | | |

### Reference Example 2

### Preparation of Drug Solution

Chitosan solution was prepared in a similar manner to that described for Example 1 above, using 20 mM histidine buffer (pH 6.5). A 26.68 mg aliquot of chitosan glutamate was dissolved in 2 mL of buffer to prepare a chitosan solution.

The chitosan solutions were then used to prepare chitosan coated VLPs by following the steps below:
(1) a container was filled with aliquots of 1.673 mL of chitosan solution,
(2) 800 μg of GI VLPs (180 μL) and 800 μg of GII VLPs (147 μL) respectively were also added,
(3) the resulting solution of total 2 mL was then mixed using a vortex mixer.

This led to a ratio of chitosan to VLPs in the formulation of 16.7.

### Preparation of Positive Control

Positive control drug solutions were prepared by following the steps below:
(1) a container was filled with 1000 μg of GI VLPs and 1000 μg of GII VLPs respectively,
(2) a 25 mg aliquot of aluminum hydroxide gel (alum) was added to the container,
(3) a 2.5 mg aliquot of monophosphoryl lipid A (MPL) was added to the container,
(4) 20 mM histidine buffer was added to make the final volume up to 2.5 mL,
(5) the mixture was mixed using a vortex mixer.

### Animal Testing

A 50 μL aliquot of each formulation was directly administered to female balb/c mice (n=4) on day 0 and day 22 either orally or via injection to the jejunum. The positive control was injected intramuscularly (IM) to female balb/c mice (n=4) on day 0 and day 22.

On day 29 of the study, serum and feces samples were collected. GI VLP-specific serum and fecal Immunoglobulin A (IgA) were measured using ELISA (Enzyme Linked Immunosorbent Assay), as described in the test methods section.

TABLE 8
Serum IgA and fecal IgA for samples tested in mice in Reference Example 2

| | | | GI | GII | Chitosan | | | | Serum IgA titer | | | Fecal IgA titer | | |
|---|---|---|---|---|---|---|---|---|---|---|---|---|---|---|
| | | Admin. | VLPs | VLPs | glutamate | Alum | MPL | Vol. | (OD_{450 nm}) | | | (OD_{450 nm}) | | |
| | Buffer | Route | (μg) | (μg) | (μg) | (μg) | (μg) | (μL) | Mean | Min | Max | Mean | Min | Max |
| Drug | 20 mM | Intra- | 20 | 20 | 667 | — | — | 50 | 433.8 | 0.0 | 1279 | 37.8 | 0.0 | 93.6 |
| substance | histidine | jejunum | | | | | | | | | | | | |
| | buffer | | | | | | | | | | | | | |
| | (pH 6.5) | | | | | | | | | | | | | |
| Drug | 20 mM | Oral | 20 | 20 | 667 | — | — | 50 | 6.2 | 0.0 | 24.9 | 0.1 | 0.0 | 0.5 |
| substance | histidine | | | | | | | | | | | | | |
| | buffer | | | | | | | | | | | | | |
| | (pH 6.5) | | | | | | | | | | | | | |
| Positive | 20 mM | Intra- | 20 | 20 | — | 50 | 500 | 50 | 204.6 | 97.5 | 368.3 | 0.5 | 0.0 | 0.8 |
| control | histidine | muscular | | | | | | | | | | | | |
| | buffer | | | | | | | | | | | | | |
| | (pH 6.5) | | | | | | | | | | | | | |

1. An aqueous composition comprising:
water,
virus like particles (VLPs),
at least one amino group containing mucoadhesive, and
at least one buffer optionally with a pH of less than about 6,
wherein the virus like particles have a size average of no more than about 4 times the size average of a comparison sample of virus like particles without said mucoadhesive in water, and/or
wherein the virus like particles have a size average of no more than about 400 nm.

2. An aqueous composition according to claim 1, wherein the at least one amino group containing mucoadhesive has a pKa of less than about 7, and
wherein the pH of the aqueous composition is at least about 1 pH unit more acidic than the value of the pKa of the amino group containing mucoadhesive.

3. An aqueous composition according to claim 1, wherein the virus like particles have a size average of no more than about 3 times the size average of a comparison sample of virus like particles without said mucoadhesive in water, or no more than about 2.5 times the size average of a comparison sample of virus like particles without said mucoadhesive in water.

4. An aqueous composition according to claim 1, wherein the virus like particles have a size average of from about 10 nm to about 350 nm, or from about 10 nm to about 300 nm, or from about 10 nm to about 250 nm or from about 10 nm to about 200 nm, or from about 10 nm to about 170 nm, or from about 10 nm to about 150 nm, or from about 10 nm to about 120 nm.

5. An aqueous composition according to claim 1, wherein the virus like particles have a positive zeta potential.

6. An aqueous composition according to claim 5, wherein the virus like particles have a positive zeta potential of at least about 15 mV, or at least about 20 mV, or at least about 30 mV.

7. An aqueous composition according to claim 1, wherein the concentration of mucoadhesive is greater than about 8 mg/mL, or greater than about 10 mg/mL, or greater than about 12.5 mg/mL.

8. An aqueous composition according to claim 1, wherein the concentration of the virus like particles (VLPs) in the aqueous composition is at least about 50 μg/mL, or is at least about 100 μg/mL, or is at least about 250 μg/mL, or is at least about 500 μg/mL, or is at least about 600 μg/mL, or is at least about 700 μg/mL, or is at least about 800 μg/mL.

9. An aqueous composition according to claim 1, wherein the weight ratio of mucoadhesive to VLPs is from about 0.5 to about 300, or from about 0.5 to about 100, or from about 10 to about 100, or from about 20 to about 100, or from about 30 to about 100, or from about 40 to about 100, or from about 10 to about 60, or from about 30 to about 60, or from about 40 to about 60, such as about 50.

10. An aqueous composition according to claim 1, wherein the concentration of the buffer is from about 25 mM to about 2000 mM, or from about 50 mM to about 1000 mM, or from about 50 mM to about 500 mM.

11. An aqueous composition according to claim 10, wherein the concentration of the buffer is from about 50 mM to about 500 mM, or from about 200 mM to about 500 mM.

12. An aqueous composition according to claim 1, wherein the buffer is acetate buffer.

13. An aqueous composition according to claim 1, wherein the mucoadhesive is selected from the group of polysaccharides (carbohydrate polymers), derivatized polysaccharides (derivatized carbohydrate polymers), cross-linked derivatives of poly(acrylic acid), derivatized polyvinyl alcohol, derivatized polyvinyl pyrollidone, polypeptides, derivatized polypeptides, proteins, derivatized proteins, derivatized deoxyribonucleic acid and polyethylenimine.

14. An aqueous composition according to claim 13, wherein the mucoadhesive is a polysaccharide, namely chitosan, wherein the chitosan is optionally admixed as chitosan base or chitosan salt.

15. An aqueous composition according to claim 14, wherein the admixed mucoadhesive is selected from the group of chitosan glutamate, chitosan hydrochloride, chitosan acetate, chitosan lactate, chitosan alginate, chitosan ascorbate, trimethylated chitosan, methyl glycol chitosan and carboxymethyl chitosan.

16. An aqueous composition according to claim 15, wherein the admixed mucoadhesive is chitosan glutamate.

17. An aqueous composition according to claim 14, wherein the weight ratio of admixed chitosan glutamate, or an equimolar amount of another type of chitosan salt or chitosan base, to VLPs is from about 30 to about 100, or from about 40 to about 60, preferably about 50.

18. An aqueous composition according to claim 1, wherein the VLPs are selected from the group of species of Norovirus VLPs, Rotavirus VLPs, HPV VLPs, Influenza virus VLPs, dengue fever virus VLPs and hepatitis B virus VLPs.

19. An aqueous composition according to claim 1, wherein the composition comprises VLPs derived from at least 1 species of virus, or VLPs derived from at least 2 different species of virus, or VLPs derived from at least 3 different species of virus, or VLPs derived from at least 4 different species of virus.

20. An aqueous composition according to claim 18, wherein the composition comprises one or more different genotypes of Norovirus VLPs selected from the group consisting of Norovirus group 1 (GI) VLPs and Norovirus group 2 (GII) VLPs.

21. An aqueous composition according to claim 1, wherein the pH of the buffer is from about 2.5 to about 5.5, or about 3.0 to about 5.0 or about 3.5 to about 4.5.

22. An aqueous composition according to claim 1, wherein the pH of the solution is at least about 1 pH unit more acidic than the pKa of the mucoadhesive, or is at least about 1.5 pH units more acidic than the pKa of the mucoadhesive, or is at least about 2.0 pH units more acidic than the pKa of the mucoadhesive, or is at least about 2.5 pH units more acidic than the pKa of the mucoadhesive, or is at least about 3.0 pH units more acidic than the pKa of the mucoadhesive.

23. An aqueous composition according to claim 1, wherein the pH of the solution is from about 2.5 to about 5.5 and preferably, the mucoadhesive is chitosan.

24. An aqueous composition according to claim 23, wherein the pH of the solution is about 5.5 or less, or about 5 or less, or about 4.5 or less, or about 4 or less and preferably the mucoadhesive is chitosan.

25. An aqueous composition according to claim 24, wherein the composition has a pH of from about 2.5 to about 5, or about 2.5 to about 4.5, such as about pH 4 or about pH 3 and preferably the mucoadhesive is chitosan.

26. An aqueous composition according to claim 25 comprising:
water,
virus like particles,
chitosan, and
at least one buffer with a pH of from about 2.5 to about 5,
wherein the virus like particles are Norovirus virus like particles;
wherein the virus like particles have a size average of no more than about 400 nm, and
wherein, the weight ratio of admixed chitosan glutamate, or an equimolar amount of another type of chitosan salt or chitosan base, to VLPs is from about 30 to about 100, or from about 40 to about 60, preferably about 50.

27. An aqueous composition according to claim 1, wherein a further adjuvant, such as alum or MPL, is not included in the composition.

28. A dry composition with a residual water content of less than about 5% w/w, or less than about 4% w/w, or less than about 2% w/w, or less than about 1% w/w, or less than about 0.1% w/w obtainable by drying the aqueous composition according to claim 1.

29. (canceled)

30. (canceled)

31. (canceled)

32. A method of eliciting protective immunity to a viral infection in a mammal comprising administering to a mammal in need of such a treatment a composition according to claim 1.

33. A method of eliciting protective immunity to a viral infection in a mammal comprising mucosally administering to a mammal in need of such a treatment a composition according to claim 1.

34. A method of eliciting protective immunity to a viral infection in a mammal comprising orally administering to a mammal in need of such a treatment a composition according to claim 1.

35. (canceled)

36. (canceled)

37. (canceled)

38. (canceled)

39. (canceled)

40. (canceled)

41. A process of preparing an aqueous composition comprising:
water,
virus like particles,
at least one amino group containing mucoadhesive, and
at least one buffer optionally with a pH of less than about 6,
wherein the virus like particles have a size average of no more than about 4 times the size average of a comparison sample of virus like particles without said mucoadhesive in water, and/or
wherein the virus like particles have a size average of no more than about 400 nm, the process comprising at least the following steps:
(a) providing virus like particles,
(b) providing at least one amino group containing mucoadhesive in a buffered aqueous composition with at least one buffer, optionally with a pH of less than about 6, to obtain a mucoadhesive containing aqueous composition,
(c) adding the virus like particles to the mucoadhesive containing aqueous composition.

42. The process according to claim 41,
wherein the at least one amino group containing mucoadhesive has a pKa of less than about 7,
wherein the pH of the aqueous composition is at least about 1 pH unit more acidic than the value of the pKa of the mucoadhesive, and
wherein step (b) involves providing at least one amino group containing mucoadhesive in a buffered aqueous composition with a pH of at least about 1 pH unit more acidic than the value of pKa of the mucoadhesive, to obtain a mucoadhesive containing aqueous composition.

43. The process according to claim 41, wherein the mucoadhesive added in step (b) is chitosan, such as chitosan base or chitosan salt.

44. A product obtainable by the process of claim 41.
